(19) United States
(12) Patent Application Publication
(10) Pub. No.: US 2005/0119281 A1
(43) Pub. Date: Jun. 2, 2005

(54) Powder composition comprising zaleplon of defined particle size distribution and pharmaceutical products made therefrom

(75) Inventors: Erika Feher, Debrecen (HU); Ferenc Korodi, Debrecenc (HU); Claude Singer, Kfar Saba (IL); Czaba Szabo, Debrecen (HU); Judith Aronhime, Rehovot (IL); Sheldon Deck, Petah-Tiqva (IL)

Correspondence Address: KENYON & KENYON, ONE BROADWAY, NEW YORK, NY, 10004, US

(73) Assignee: Biogal Gyogyszergyar Rt, Debrecen (HU)

(21) Appl. No.: 10/504,263

(22) PCT Filed: Feb. 19, 2003
(86) PCT No.: PCT/US03/07267

Related U.S. Application Data
(60) Provisional application No. 60/357,552, filed on Feb. 15, 2002.

(51) Int. Cl. A61K 31/519; A61L 9/04; A61K 9/14

(52) U.S. Cl. 514/262.1; 424/46

(57) ABSTRACT

The present invention provides a powder composition comprising zaleplon of defined particle size distribution. The zaleplon of defined particle size can be formulated into a wide variety of pharmaceutical compositions and dosage forms.

### CROSS-REFERENCE TO RELATED APPLICATION

This application claims the benefit of provisional application Ser. No. 60/357,552, filed Feb. 15, 2002, which is incorporated herein by reference.

### FIELD OF THE INVENTION

The present invention relates to zaleplon of defined particle size distribution.

### BACKGROUND OF THE INVENTION

Zaleplon, whose systematic chemical name is N-[3-(3-cyanopyrazolo[1,5-a]pyrimidin-7-yl)phenyl]-N-ethylacetamide, possesses anxiolytic, antiepileptic, sedative and hypnotic properties. It is approved by the U.S. Food and Drug Administration for short-term treatment of insomnia.

Zaleplon and a process for preparing it are disclosed in U.S. Pat. No. 4,626,538, which is incorporated herein by reference.

Particle size can affect the solubility properties of a compound like zaleplon. Particle size reduction may be tried in order to increase a compound's solubility. Particle size reduction increases the surface area of the solid phase that is in contact with the liquid medium. However, particle size reduction cannot alter the solubility of the compound in a solvent, which is a thermodynamic quantity.

There are instances where the rate of dissolution of a poorly soluble drug is the rate limiting factor in its rate of absorption by the body. It is recognized that such drugs may be more readily bioavailable if administered in a finely divided state.

Particle size also can affect how freely crystals or a powdered form of a drug will flow past each other which has consequences in the production process of pharmaceutical products containing the drug.

In view of the foregoing, there is a need in the medical arts for zaleplon with defined particle size distribution.

### SUMMARY OF THE INVENTION

The present invention provides a powder composition of zaleplon having a defined particle size distribution. In accordance with this invention, zaleplon particles of the composition have a particle size distribution in which 10% or fewer of the particles have a diameter below about 0.5 μm, 10% or fewer of the particles have a diameter above about 20 μm, and the median particle diameter is from about 4 to about 10 μm.

The invention further provides pharmaceutical compositions and dosage forms made from the powder composition and methods of treating insomnia by administering the pharmaceutical compositions and dosage forms of the invention.

### DESCRIPTION OF THE PREFERRED EMBODIMENTS

The present invention provides a powder composition comprising a plurality of zaleplon particles in a pre-determined size distribution. This composition is useful for preparing compressed solid dosage forms, encapsulated free flowing dosage forms, enteral solutions, suspensions and elixirs and parenteral solutions.

The powder composition of this invention comprises a plurality of zaleplon particles. Particles of the plurality will vary in characteristics and the characteristics of no individual or small proportion of the particles will materially affect the properties of the bulk material. Rather, the characteristics of the zaleplon are determined from a statistically significant sampling and measurement of bulk properties of the sample. Statistically significant measurements include those with a statistical sampling error of about 2% or less.

A “powder composition” means a powder that consists entirely of zaleplon or that contains zaleplon in intimate or non-intimate mixture with one or more other substances. A “pharmaceutical composition,” as used herein, means a medicament for use in treating a mammal that comprises zaleplon prepared in a manner that is appropriate for administration to a mammal. A pharmaceutical composition also may contain one or more pharmaceutical excipients that are non-toxic to the mammal intended to be treated when the composition is administered in an amount effective to treat the mammal. A pharmaceutical composition includes feedstocks for preparing pharmaceutical dosage forms such as tablets and capsules and medicaments that are purchased by the consumer in undivided dosages such as suspensions, syrups and solutions.

As used herein, the term “median,” when used in reference to the size of zaleplon particles, indicates that about 50% of all measurable particles measured have a particle size less than the defined median particle size value, and that about 50% of all measurable particles measured have a particle size greater than the defined median particle size value.

In accordance with the invention, the pharmaceutical composition comprises a plurality of zaleplon particles having a particle size distribution in which 10% or fewer of the particles have a diameter below about 0.5 μm, 10% or fewer of the particles have a diameter above about 20 μm, and the median particle diameter is from about 4 to about 10 μm.

In accordance with the invention, the size distribution of zaleplon particles is determined by laser diffraction. Our method of determining the size of zaleplon particles used a Malvern™ Mastersizer laser diffraction instrument. Samples of the zaleplon were suspended in hexane containing a surfactant, 1% Tween® 80. The suspensions were mixed and then sonicated for 120 seconds to thoroughly disperse the zaleplon particles. The dispersion was then circulated in the flow cell of the Malvern Mastersizer for two minutes before particle size measurements were taken.

Zaleplon of defined particle size may be produced by precipitation from appropriate solvents. Particle size may be adjusted by customary methods such as cooling, pH adjustment, pouring a concentrated solution into an anti-solvent and/or by co-precipitation so as to obtain a precipitate with the appropriate particle size distribution.

Zaleplon of defined particle size may be produced by known methods of particle size reduction starting with crystals, powder aggregates and course powder of either crystalline or amorphous zaleplon. The principal operations of conventional size reduction are milling of a feedstock material and sorting of the milled material by size.

A fluid energy mill, or micronizer, is an especially preferred type of mill for its ability to produce particles of small size in a narrow size distribution. The feedstock should be provided in an average particle size range of about 150 to 850 μm which may be achieved using a conventional ball, roller or hammer mill if necessary. As those skilled in the art are aware, fluid energy mills use the kinetic energy of collision between particles suspended in a rapidly moving fluid (typically air) stream to cleave the particles. The suspended particles are injected under pressure into a recirculating gas stream. Smaller particles are carried aloft inside the mill and swept into a vent and are collected. The vent may be connected to a particle size classifier such as a cyclone. Fluid energy mills are designed so that particles are classified by mass. Only particles with a momentum in a certain range will enter the vent and be collected. Centrifugal forces serve to classify the particles in a fluid energy mill. When milled in another type of mill, a powder composition according to this invention can be produced using cyclonic or centrifugation separation techniques.

In accordance with the invention, the powder composition comprises zaleplon of defined particle size and optionally one or more other substances, such as pharmaceutical excipients. The powder composition of this invention may be formulated into a variety of solid and liquid dosage forms for administration to humans and animals. The dosage forms include those suitable for enteral (oral, sublingual, buccal, rectal) administration. Dosage forms include solid dosage forms like tablets, powders, capsules, suppositories, sachets, troches and losenges. The most suitable route in any given case will depend on the nature and severity of the condition being treated and other circumstances that will be assessed by the caregiver.

The powder composition may be made into a solid oral dosage form such as a tablet. For making a tablet, it will typically be desirable to include one or more benign pharmaceutical excipients in the composition. The powder composition of the present invention may contain one or more diluents added to make the tablet larger and, hence, easier for the patient and caregiver to handle. Common diluents are microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Binders also may be included to help hold the tablet together after compression. Some typical binders are acacia, alginic acid, carbomer (e.g. carbopol), carboxymethylcellulose sodium, dextrin, ethyl cellulose, gelatin, guar gum, hydrogenated vegetable oil, hydroxyethyi cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

The tablet may further include a disintegrant to accelerate disintegration of the tablet in the patient's stomach. Disintegrants include alginic acid, carboxymethyl cellulose calcium, carboxymethylcellulose sodium (e.g. Ac-Di-Sol®, Primellose®), colloidal silicon dioxide, croscarmellose sodium, crospovidone (e.g. Kollidone®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

A powder composition for tableting may further include glidants, lubricants, flavorings, colorants and other commonly used excipients.

Liquid oral pharmaceutical compositions of the present invention are produced from the powder composition containing zaleplon of defined particle size distribution and a liquid carrier such as water, vegetable oil, alcohol, polyethylene glycol, propylene glycol or glycerin, most preferably water.

Liquid oral pharmaceutical compositions may contain emulsifying agents to disperse uniformly throughout the composition the active ingredient or other excipient that has low solubility in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid oral pharmaceutical compositions of the present invention may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

The liquid oral pharmaceutical composition also may contain sweetening agents, such as sorbitol, saccharin, sodium saccharin, sucrose, aspartame, fructose, mannitol and invert sugar; preservatives and chelating agents such as alcohol, sodium benzoate, butylated hydroxy toluene, butylated hydroxyanisole and ethylenediamine tetraacetic acid; and buffers such as guconic acid, lactic acid, citric acid or acetic acid, sodium guconate, sodium lactate, sodium citrate or sodium acetate.

For insomnia therapy involving oral administration of a solid or liquid dosage form, an orally administered unit dosage contains normally from 5 to 20 mg, more preferably about 10 mg.

### EXAMPLE

Zaleplon as received was recrystallized from ethanol and water and dried under vacuum. The particle size distribution of the crystals was analyzed and it was found that 10% of the particles were equal to or less than 14 μm in diameter, the median particle size was 55 μm and 90% of particles had a diameter of 134 μm or less.

The recrystallized zaleplon was milled in a conical, continuous operation mill. The milled zaleplon had a particle size distribution in which 10% of the particles had a diameter of 8.3 μm or less, the median particle size was 37.0 μm and 90% of the particles had a diameter of 84.6 μm or less.

The micronized zaleplon was used as a feedstock for a fluid energy mill. The targeted particle size distribution was controlled by adjusting the feed rate, the pressure of the feed air and the pressure of the grinding air. The micronized zaleplon had a particle size distribution in which the median particle size was 6.3 μm, and 90% of the particles had a diameter of 15.1 μm or less.

Having thus described the invention with respect to certain preferred embodiments and further illustrated it with examples, those skilled in the art may come to appreciate substitutions and equivalents that albeit not expressly described are taught or inspired by this invention. Whereas such substitutions and equivalents do not depart from the spirit of the invention they are within its scope which is defined by the claims which follow.

1. A powder composition comprising a plurality of zaleplon particles having a particle size distribution in which 10% or fewer of the particles have a diameter below about 0.5 μm, 10% or fewer of the particles have a diameter above about 20 μm, and the median particle diameter is from about 4 to about 10 μm.

2. A powder composition of claim 1 that consists essentially of zaleplon.

3. A powder composition of claim 2 that consists of zaleplon.

4. A powder composition of claim 1 prepared by milling a zaleplon feedstock of 150-850 μm median particle size in a fluid energy mill.

5. A dosage form comprising the powder composition of claim 1.

6. A solid oral dosage form of claim 5.

7. The solid oral dosage form of claim 6 wherein the composition comprises at least one benign pharmaceutical excipient.

8. The solid oral dosage form of claim 7 wherein the at least one benign pharmaceutical excipient is selected from the group consisting of microcrystalline cellulose, microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylate, potassium chloride, powdered cellulose, sodium chloride, sorbitol, talc, acacia, alginic acid, carbomer, carboxymethylcellulose sodium, dextrin, ethyl cellulose, gelatin, guar gum, hydrogenated vegetable oil, hydroxyethyl cellulose, hydroxypropyl cellulose, hydroxypropyl methylcellulose, liquid glucose, magnesium aluminum silicate, maltodextrin, methylcellulose, polymethacrylates, povidone, pregelatinized starch, sodium alginate, starch, alginic acid, carboxymethyl cellulose calcium, colloidal silicon dioxide, croscarmellose sodium, crospovidone, guar gum, magnesium aluminum silicate, methyl cellulose, polacrilin potassium, powdered cellulose, pregelatinized starch, sodium alginate and sodium starch glycolate.

9. The solid oral dosage form of claim 6 containing a unit dose of from about 5 to abut 20 milligrams of zaleplon.

10. A method of treating insomnia comprising administering a dosage form of claim 5.

11. A liquid pharmaceutical composition prepared by contacting the powder composition of claim 1 and a liquid carrier.

12. The liquid pharmaceutical composition of claim 11 wherein the liquid carrier is selected from the group consisting of water, vegetable oil, alcohol, polyethylene glycol, propylene glycol and glycerin.

13. The liquid pharmaceutical composition of claim 12 wherein the liquid carrier is water.

14. The liquid pharmaceutical composition of claim 11 further comprising at least one excipient selected from the group consisting of gelatin, egg yolk, casein, cholesterol, acacia, tragacanth, chondrus, pectin, methyl cellulose, carbomer, cetostearyl alcohol, cetyl alcohol, alginic acid bentonite, carbomer, carboxymethylcellulose calcium or sodium, ethylcellulose, gelatin guar gum, hydroxyethyl cellulose, hydroxypropyl cellulose, hydroxypropyl methylcellulose, maltodextrin, polyvinyl alcohol, povidone, propylene carbonate, propylene. glycol alginate, sodium alginate, sodium starch glycolate, starch tragacanth, xanthan gum, sorbitol, saccharin, sodium saccharin, sucrose, aspartame, fructose, mannitol, invert sugar; ethyl alcohol, sodium benzoate, butylated hydroxy toluene, butylated hydroxyanisole, ethylenediamine tetraacetic acid, guconic acid, lactic acid, citric acid, acetic acid, sodium guconate, sodium lactate, sodium citrate and sodium acetate.

15. A method of treating insomnia comprising administering a liquid pharmaceutical composition of claim 11.
